(19) United States
(12) Patent Application Publication
(10) Pub. No.: US 2024/0017029 A1
(43) Pub. Date: Jan. 18, 2024

(54) ENDOTRACHEAL TUBE

(71) Applicant: BON SECOURS MERCY HEALTH, INC., Cincinnati, OH (US)

(72) Inventors: Anthony D’Errico, Meridian, ID (US); David Michael Uhlenhake, Toledo, OH (US)

(21) Appl. No.: 18/252,159

(22) PCT Filed: Nov. 8, 2021
(86) PCT No.: PCT/US21/58385

§ 371 Date: May 8, 2023

Related U.S. Application Data
(60) Provisional application No. 63/110,439, filed on Nov. 6, 2020.

(51) Int. Cl. A61M 16/04; A61B 1/267

(52) U.S. Cl. CPC A61M 16/0486; A61B 1/267; A61M 16/0484; A61M 2209/10

(57) ABSTRACT

An endotracheal tube includes a tube body. The tube body can have a distal end configured to be disposed in an airway of a patient and a proximal end configured to remain outside the airway. The tube body can include a first tube and a second tube. The first tube can be configured to be in communication with a ventilator and the second tube can be configured to receive a visualization device. The visualization device can be configured to be selectively advanced along a length of the second tube. A cleaning system can be disposed in the second tube. A flexible tip member can be disposed on the distal end of the tube body.

### CROSS REFERENCE TO RELATED APPLICATIONS

This application claims the benefit of International Patent Application No. PCT/US21/58385 filed on Nov. 8, 2021, which claims the benefit and priority of U.S. Provisional Application Ser. No. 63/110,439, filed on Nov. 6, 2020. The entire disclosures of the above applications are incorporated herein by reference.

### FIELD

The present technology relates to respiratory tubes, and more particularly, endotracheal tubes.

### INTRODUCTION

This section provides background information related to the present disclosure which is not necessarily prior art.

Respiratory tubes, including endobronchial tubes, endotracheal tubes, and tracheostomy tubes, are used to ventilate at least a portion of the respiratory system or lungs of a subject. Such respiratory tubes may be inserted in a number of ways via a non-invasive approach through an orifice or cavity, such as the oral or nasal cavity. Alternatively, such respiratory tubes may be introduced to a body via a minimally invasive external incision creating a port for tube insertion, for example, through the trachea in a tracheotomy procedure.

Certain respiratory tubes may be provided as single lumen or double lumen tubes for selectively ventilating a portion of the respiratory system. For example, endobronchial tubes, whether single lumen or double lumen, may be utilized for one-lung ventilation procedures or for selective lung ventilation of the left or right bronchi during one-lung ventilation procedures. However, respiratory tube designs can present limitations in use, where certain tubes are not conducive to emergency settings, certain tubes can be cumbersome and require additional equipment to install, and certain tubes are designed only for a single use or procedure.

During an intubation procedure, an endotracheal tube can be placed in a patient who is unable to effectively maintain life-sustaining ventilation and respiration on their own. Use of an endotracheal tube is appropriate when the integrity of the airway is, or may become, challenged due to trauma or pathology, or if a patient cannot otherwise breathe unaided. Often the endotracheal tube is coupled to a mechanical ventilator to aid the patient's respiration, and can be expected to remain in situ for an extended time until the patient is once again able to breathe on his or her own. The endotracheal tube can be inserted within a patient's native airway for short periods of time (e.g., for a matter of hours during anesthesia for surgery) or the endotracheal tube can remain in place to provide ventilator-assisted breathing for days or weeks.

The institution of mechanical ventilation can result in increased production of secretions within the patient's native airway. The insertion of an endotracheal tube within the patient's airway removes the normal cough mechanism for clearing of secretions. The secretions, therefore, can pool in dependent portions of the lung over time due to gravity and, if not removed in a timely manner, can result in ventilator-acquired pneumonia (VAP) or other undesired conditions or ailments. Because the cough mechanism is not intact, invasive removal is required to remove the pooled secretions with a suction catheter.

The insertion of endotracheal tubes is often performed in emergency situations, which can result in inappropriate placement of the endotracheal tube. Inappropriate placement of the endotracheal tube within the patient can result in severe clinical compromise of the intubated patient and, in some instances, can lead to severe injury or death if not corrected. The most commonly reported errors in positioning include positioning the tube in the esophagus, introducing the endotracheal tube beyond the carina with the tip in the right main stem bronchus, and not introducing the tube far enough such that the endotracheal tube balloon is inflated at the level of the vocal cords or just proximal thereto.

Accordingly, there is a continuing need for an endotracheal tube configured for an emergency room setting, which allows for placement without a laryngoscope, and for continued intubation.

### SUMMARY

In concordance with the instant disclosure, an endotracheal tube configured for an emergency room setting, which allows for placement without a laryngoscope, and allows for continued intubation, has surprisingly been discovered.

The present technology can be a combined laryngoscope and endotracheal tube. The endotracheal tube can allow for visualization and navigation through a patient's airway. The endotracheal tube eliminates the use of a separate visualization tool such as a glide scope or laryngoscope.

In one embodiment, an endotracheal tube can be configured to be inserted in an airway of a patient and cooperate with a ventilator and a visualization device. The endotracheal tube can include a tube body. The tube body can have a distal end configured to be disposed in the airway and a proximal end configured to remain outside the airway. The tube body can further include a first tube and a second tube. The first tube can be configured to be operably coupled to the ventilator and the second tube can be configured to operably receive and have the visualization device advanced therethrough. A cleaning system can be disposed in the second tube. A flexible tip member can be disposed on the distal end of the tube body.

In another embodiment, a method of utilizing an endotracheal tube, which is configured to be inserted in an airway of a patient and cooperate with a ventilator includes providing the endotracheal tube. The endotracheal tube can include a tube body. The tube body can have a distal end configured to be disposed in the airway and a proximal end configured to remain outside the airway. The tube body can further include a first tube and a second tube. The first tube can be configured to be operably coupled to the ventilator and the second tube can be configured to operably receive and have the visualization device advanced therethrough. A cleaning system can be disposed in the second tube. A flexible tip member can be disposed on the distal end of the tube body. The endotracheal tube can be inserted into the airway. The endotracheal tube can be secured at a desired location in the airway. The visualization device can be cleaned with the cleaning system.

The endotracheal tube may be a double lumen tube that has a compact built-in flexible endoscope near an end of the tube. This allows for visualization of an airway when swelling, secretions, or sub-optimal anatomy obscure an operator's view of the glottic structures. This is achieved without having the need for additional manipulation or suction tools that further crowd an already limited working space.

The endotracheal tube may further aid in troubleshooting tube integrity, tube placement, and distal airway plugging. Further, obstruction can easily be assessed with bronchoscope features, which may be employed by advancing the scope. The scope is equipped with standard bronchoscope features, including irrigation and suction. The scope may be further utilized by advancing scope towards distal airways.

More particularly, the device is configured to be used to entire time a patient is intubated, as opposed to being configured for a single procedure. Accordingly, the tube of the endotracheal tube may be more flexible near a distal end of the device. Further, the distal end may have openings formed therein to improve the flexibility of the end. The tube may have a gradient flexibility, where the tube may be more flexible when moving from a proximal end to the distal end. The endotracheal tube may also include a cleaning mechanism. The cleaning mechanism may include a tapered end and space between the wall of the tube and the camera to allow a water film to be disposed on the lens of the camera.

In one embodiment, an endotracheal tube can be a double lumen tube. The endotracheal tube can be fabricated from a flexible material. The flexible material should be safe for insertion into a patient, flexible enough to maneuver an airway of the patient, but firm enough to retain its shape within the airway. The flexible material can be a plastic material such as polyvinyl chloride (PVC), as a non-limiting example. A skilled artisan can select other suitable materials for the endotracheal tube within the scope of the present disclosure.

The endotracheal tube can have a distal end and a proximal end. The distal end can be configured to be disposed in the airway of the patient. The proximal end can be configured to remain outside of the airway of the patient and to communicate with equipment, for example, a ventilator. It should be appreciated that the endotracheal tube according to the present technology can be configured to be used through an entire intubation of the patient, as opposed to being configured to be used through a single procedure. Additionally, the endotracheal tube can be configured to be inserted into the airway of the patient without the use of additional tools, for example, a laryngoscope. Accordingly, the distal end can be sufficiently flexible to allow for intubation and the proximal end can be sufficiently rigid to be used throughout intubation. In other words, the endotracheal tube can have a gradient flexibility along a length of the endotracheal tube, in which, the tube can become more rigid moving from the distal end to the proximal end.

In order to increase the flexibility of the distal end of the endotracheal tube, a hole can be formed adjacent to the distal end. More particularly, a plurality of holes can be formed around the exterior surface of the distal end of the endotracheal tube. Advantageously, the plurality of holes provides additional flexibility at the distal end, while also militating against the distal end of the endotracheal tube tracheal tube from becoming blocked within the airway of the patient.

A visualization device can be disposed near the distal end of the endotracheal tube. The visualization device can be configured to be used during placement of the endotracheal tube and during intubation to monitor the airway of the patient for diagnostic purposes. This allows for visualization of the airway of the patient when swelling, secretions, or sub-optimal anatomy obscure an operator's view of the glottic structures. Advantageously, this can be achieved without having the need for additional manipulation or suction tools that further crowd an already limited working space. The visualization device can be a camera or an endoscope, as non-limiting examples.

The endoscope can be equipped with bronchoscope features such as a light, a suction device, and an irrigation device. These bronchoscope features can allow a clinician to more clearly visualize the airway of the patient, in operation. The visualization device can be disposed within the endotracheal tube, or the visualization device can be disposed in a sleeve disposed on an exterior surface of the endotracheal tube.

The visualization device can be slidably disposed near the distal end of the endotracheal tube. Accordingly, the visualization device can be selectively forwarded out of the distal end of the endotracheal tube. Advantageously, the slidable function of the visualization device can allow the clinician to visualize the airway of the patient beyond a length of the endotracheal tube, which can improve the diagnostic function of the endotracheal tube.

The endotracheal tube can further include a cleaning system for the visualization device. The cleaning system can be configured to remove buildup of debris on the visualization device, which can otherwise prevent the visualization device from normal operation within the airway of the patient. The cleaning device can also militate against the formation of debris on the visualization device. In particular, the cleaning system can be configured to form a water layer on an exterior surface of the visualization device. The water layer can remove existing debris, while also militating against the formation of additional debris.

The distal end of the endotracheal tube can be tapered to house the cleaning system. The visualization device can be spaced apart from an interior wall of the endotracheal tube. The cleaning system can include a water tube disposed adjacent to the distal end of the endotracheal tube, which is configured to deposit the water layer on the visualization device. The water tube can be disposed between the visualization device and the interior wall, adjacent to a longer portion of the tapered end. A skilled artisan can select other suitable components for the cleaning system, as required.

It should be appreciated that the endotracheal tube can be configured to work with a removeable stylet. The stylet can aid in placement of the endotracheal tube, in operation, as desired. The endotracheal tube is primarily navigated through the airway by the reusable stylet. The stylet can allow the endotracheal tube to be stiffened and can mold a shape of the endotracheal tube as desired. Accordingly, the stylet alters the shape of an endotracheal tube to facilitate intubation and stiffens the endotracheal tube to aid passage into the trachea.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a side elevational view of an endotracheal tube, according to one embodiment of the present disclosure, further depicted with a stylet and a visualization device disposed therein;

FIG. 2 is an enlarged side elevational view of a flexible tip member of the endotracheal tube shown in FIG. 1,

FIG. 3 is an enlarged side elevational view of a plurality of holes of the flexible tip member of the endotracheal tube taken at callout A in FIG. 2;

FIG. 4 is a side elevational view of the endotracheal tube of FIG. 1, further depicted in use in an airway of a patient with the stylet removed;

FIG. 5 is an enlarged side elevational view of a cleaning system of the endotracheal tube taken at callout B in FIG. 4, further depicting a visualization device moving toward the cleaning system;

FIG. 6 is an enlarged side elevational view of a cleaning system of the endotracheal tube taken at callout B in FIG. 4, further depicting a visualization device moving through the cleaning system; and

FIG. 7 is a flow chart depicting a method of utilizing the endotracheal tube according to another embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The term “airway(s)” as used herein shall be given its ordinary meaning and shall include, without limitation, the oral cavity, nasal passages, pharynx, larynx, trachea, and/or any portion of the lungs, including any of the branches of the tracheobronchial tree.

The term “debris” as used herein shall include, without limitation, biological fluids, solids, gels, deposits, films, debris, and/or secretions, such as mucosal secretions, blood, and/or any other biological or biologically-related materials.

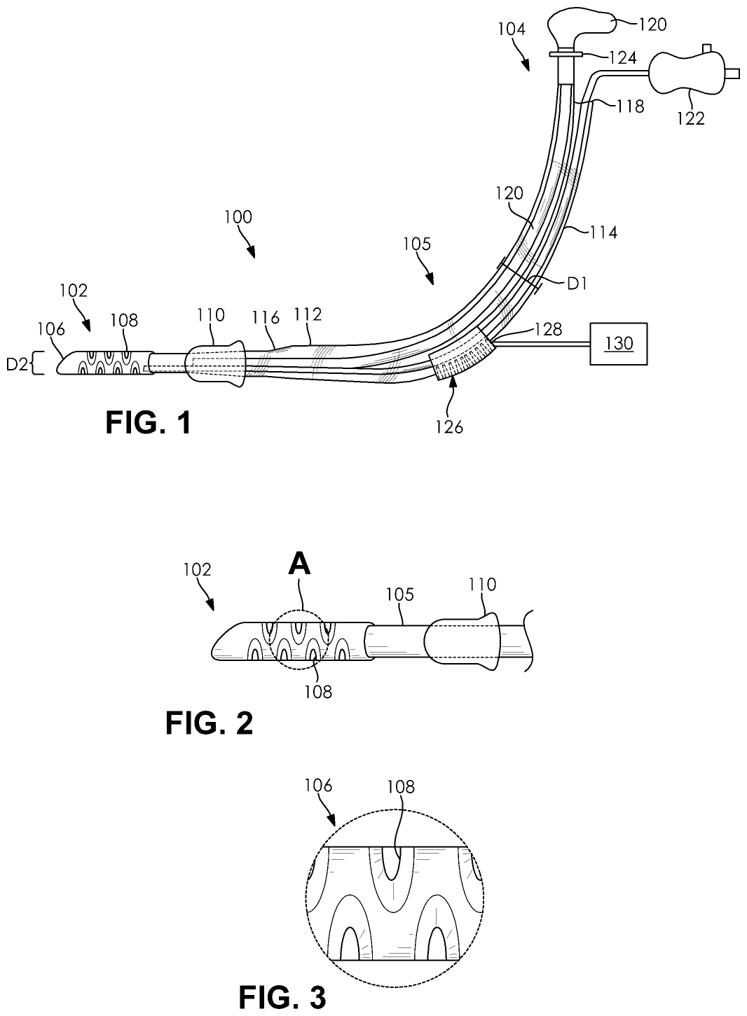

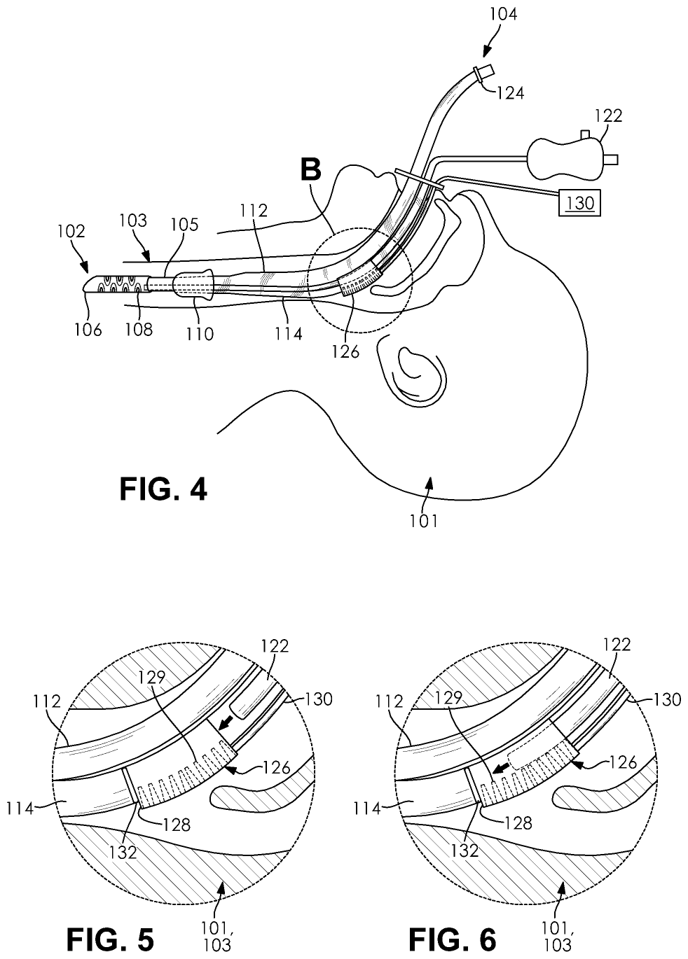

With reference to FIGS. 1-6, an endotracheal tube 100 is shown. It should be appreciated that the endotracheal tube 100 according to the present technology can be configured to be used through an entire intubation of a patient 101, as opposed to being configured to be used through a single procedure. Additionally, the endotracheal tube 100 can be configured to be inserted into an airway 103 of the patient 101 without the use of additional tools, for example, a laryngoscope. In some embodiments, the endotracheal tube 100 (or one or more of its component parts) can be intended to be a single-use, sterile or sterilizable, disposable medical device. In other embodiments, however, the endotracheal tube 100 (or one or more of its component parts) can be sterilized and reused. Further, although the endotracheal tube 100 shown throughout the figures is depicted as transparent, it should be appreciated that the endotracheal tube 100 can be opaque, as desired.

The endotracheal tube 100 can include a distal end 102 and a proximal end 104. The distal end 102 of the endotracheal tube 100 can be configured to be disposed in the airway 103 of the patient 101. The proximal end 104 of the endotracheal tube 100 can be configured to remain outside of the airway 103 of the patient 101 and to communicate with equipment, for example, a ventilator, as described in greater detail hereinbelow.

Materials used for the various components of the endotracheal tube 100 described herein can advantageously include one or more biocompatible materials. Such materials can be semi-rigid and/or flexible, as desired or required for a particular application or use. The materials used can include, but are not limited to, one or more of polyether ether ketone (PEEK), Nylon 6/6, polyethylene, polypropylene, polyethylene terephthalate (PET), glycol-modified PET, polyvinyl chloride (PVC), thermoplastic elastomers (TPEs), other natural or synthetic polymers (e.g., KRATON polymers), silicone, natural rubber, latex, polycarbonate, K resin, acrylonitrile butadiene styrene (ABS), styrenes and/or other thermoplastic elastomers or polymers. A skilled artisan can select other suitable materials for the endotracheal tube 100 within the scope of the present disclosure.

An outer diameter of the endotracheal tube 100 can be configured to fit within the airway 103 of the patient 101. For example, the outer diameter of the endotracheal tube 100 can be from about 2 millimeters to about 14 millimeters, depending on the size of the patient, including considerations for factors such as age, weight, and sex of the patient. The outer diameter of the endotracheal tube 100 can be modified based on the patient 101, as needed. Likewise, a length of the endotracheal tube 100 can depend on a size of the patient 101. For example, an adult male patient can require a longer endotracheal tube 100 than a child. A radial cross-section of the endotracheal tube 100 can be circular, substantially circular, elliptical, oval, and/or any other shape that allows for insertion into the airway 103 of the patient 101. A skilled artisan can select suitable shapes and dimensions for the endotracheal tube 100, as desired.

The endotracheal tube 100 can include a tip member 106 disposed at the distal end 102. The tip member 106 can be flexible to allow for maneuverability through the airway 103 of the patient 101. As shown in FIGS. 2-3, the tip member 106 can include a plurality of holes 108 formed therethrough. In particular, the holes 108 can be provided in an array across an exterior surface of the tip member 106. It should be appreciated that the holes 108 can increase an overall flexibility of the tip member 106. While bound to no particular theory, it is believed that the holes 108 create additional points of flexion on the flexible tip member 106. Advantageously, an increased flexibility and malleability of the tip member 106 can allow for an improved ability to navigate the airway 103 of the patient 101.

It should be further appreciated that the holes 108 can further improve the functionality of the endotracheal tube 100. In particular, in operation, the endotracheal tube 100 can come into contact with various debris in the airway 103 of the patient. This debris can interfere with oxygen delivery through the endotracheal tube 100. The holes 108 can allow for additional oxygen flow that is not available in an endotracheal tube 100 in which no holes 108 are formed. In operation, should one of the holes 108 become blocked with debris, oxygen can still flow from the remainder of the holes 108. Advantageously, the endotracheal tube 100 can be used in emergency situations without needing to clear debris from the distal end of the endotracheal tube 100, in operation. A far distal end of the tip member 106 can include a hole or aperture in line with airflow through a tube body 105 of the endotracheal tube 100, in addition to the one or more radially positioned holes 108 in the tip member 106.

The endotracheal tube 100 can include an inflatable cuff 110. The cuff 110 can be an inflatable balloon at the distal end 102 of the endotracheal tube 100. The inflatable cuff 110 can circumscribe a portion of the endotracheal tube 100 adjacent to the tip member 106 at the distal end 102 of the endotracheal tube 100. In operation, the inflated cuff 110 can produce a seal against a wall of the airway 103 of the patient 101. Advantageously, this can militate against gastric contents from entering the airway 103 and can facilitate execution of positive pressure ventilation.

The tube body 105 of the endotracheal tube 100 can include two separate tubes or lumen. In particular, the tube body 105 of the endotracheal tube 100 can include a first tube 112 and a second tube 114. Each of the first tube 112 and the second tube 114 can be provided as discrete tubes. Alternatively, each of the first tube 112 and the second tube 114 can be provided within a single tube having a divider formed therein. With reference to FIG. 1, the first tube 112 and the second tube 114 can be provided as two parallel, connected tubes, as one non-limiting example.

As shown in FIG. 1, the first tube 112 and the second tube 114 can merge at a junction 116. The junction 116 can be disposed on the endotracheal tube 100 between the proximal end 104 of the endotracheal tube 100 and the inflatable cuff 110. It should be appreciated that the junction 116 can be disposed nearer to the distal end 104 of the endotracheal tube 100 than the proximal 106 end of the endotracheal tube 100. It should be appreciated that the first tube 112 and the second tube 114 can merge to form a single tube at the junction 116. In certain embodiments, the second tube 114 can extend into the first tube 112 after the junction 116, as described in greater detail, hereinbelow. Accordingly, a diameter of the first tube 112 can be greater than a diameter of the second tube 114.

Advantageously, the junction 116 can allow for a reduced diameter of the endotracheal tube 100 at the distal end 102. In other words, the endotracheal tube 100 can have a larger outer diameter between the proximal end 104 of the endotracheal tube 100 and the junction 116 (D1) compared to the outer diameter of the endotracheal tube 100 between the distal end 102 and the junction 116 (D2). This can allow for an improved seal formed by the inflatable cuff 110, as described hereinabove, in operation. Minimizing the diameter of the endotracheal tube 100 at the distal end can further improve maneuverability of the endotracheal tube 100 through the airway 103 of the patient 101.

The first tube 112 can be configured to be in communication with a ventilator in operation. In some embodiments, the proximal end 104 of the first tube can include a coupling element 124 for connection with a mechanical ventilator. It should be appreciated that localized oxygen can be provided through the first tube 112 while insertion of the endotracheal tube 100 takes place. During insertion of the endotracheal tube 100, the first tube 112 can be configured to receive a stylet 120 (e.g., an obturator) or similar device. For example, the stylet 120 is shown in FIG. 1 inserted through the first tube 112 and past the junction 116. In some embodiments, the stylet 120 can be malleable and can be used to reshape the endotracheal tube 100, during insertion. For example, the stylet 120 can be used to alter the radius or bend angle of the distal end 102 of the endotracheal tube 100 depending on the anatomical characteristics of the patient or as desired and/or required by a patient care provider. Advantageously, the stylet 120 can improve the ability to insert the endotracheal tube 100 by providing additional rigidity to the endotracheal tube 100.

The second tube 114 can be in communication with a visualization device 122 (e.g. a scope). The visualization device 122 can be used to, among other things, verify or confirm proper positioning of the endotracheal tube 100 within the airway 103 of a patient 101, visualize an interior of the endotracheal tube 100, visualize the airway 103 of a patient 100 beyond the endotracheal tube 100, and/or for any other purpose. The visualization device 122 can provide visualization of the entire airway 103 from the mouth to the carina, as well as other locations in the patient, without requiring the use of a laryngoscope. For example, the visualization device 122 can provide visualization of one or more of the uvula, the vocal cords, the trachea, the carina, and/or the right and left main bronchi or stems of the lungs, other portions of a patient's airways and/or the like using a single device.

The visualization device 122 can be slidably, removably disposed in the second tube 114. In particular, the visualization device 122 can be disposed into an opening 118 in the proximal end 104 of the second tube 114 and can be advanced along the length of the second tube 114 from the proximal end 104 to the distal end 102. In certain embodiments, the visualization device 122 can be extended beyond the flexible tip member 106. Advantageously, the slidable function of the visualization device 122 through the second tube 114 can allow the clinician to visualize the airway 103 of the patient 101 along the entire length of the endotracheal tube 100, which can improve the diagnostic function of the endotracheal tube 100.

As described hereinabove, the second tube 114 can extend into the first tube 112 after the junction 116. The second tube 114 can provide a guide for the visualization device 122 after the junction 116. Advantageously, the second tube 114 can militate against interference between the visualization device 122 and the stylet 120, in operation.

As non-limiting examples, the visualization device 122 can include an endoscope, a bronchoscope, or a camera. The visualization device 122 can be equipped with additional features, such as a light source, a suction device, and an irrigation device. Alternatively, the visualization device 122 can be removed from the second tube 114 and replaced with another device having suction and/or irrigation operability, where the visualization device 122 and the another device can be used interchangeably. These features can allow a clinician to more clearly visualize the airway of the patient, in operation. The visualization device 122 can be in communication with an external monitor (not shown). The monitor can allow the clinician to monitor insertion and placement of the endotracheal tube 100.

The endotracheal tube 100 can further include a cleaning system 126 for the visualization device 122. The cleaning system 126 can be configured to remove buildup of debris on the visualization device 122, which can otherwise prevent the visualization device 122 from normal operation within the airway 103 of the patient 101. The cleaning system 126 can include a physical agitator 128 configured to remove debris from the visualization device 122. The physical agitator 128 can include a plurality of bristles 129 formed thereon, for example, as shown in FIGS. 5-6. The bristles can be manufactured from a synthetic material. The material can be soft enough to clean a lens of the visualization device without damage. The cleaning system can also include a fluid injection assembly 130. The fluid injection assembly 130 can be configured to provide cleaning fluid (e.g., water, saline, etc.) to the physical agitator 128 to allow for additional cleaning of the lens of the visualization device 122 as well as flushing/cleaning of the physical agitator 128. A skilled artisan can select other materials for the cleaning system 126 within the scope of the present disclosure. Advantageously, the cleaning system 126 of the present disclosure can provide for debris removal from the visualization device 122 multiple times throughout a procedure without necessitating the removal of the visualization device 122 from the endotracheal tube 100.

In operation, when the visualization device 122 has built up debris, the visualization device 122 can be retracted through the second tube 114 toward the proximal end 104. During this retraction, the visualization device 122 can pass through the physical agitator 128. The visualization device 122 can be repeatedly passed through the physical agitator 128 and fluid from the fluid injection assembly 130 be applied, as needed, until the debris is cleared from the lens.

The endotracheal tube 100 can include a sealing member 132 formed therein, where the sealing member 132 can be disposed adjacent to the cleaning system 126, as shown in FIGS. 5-6. The sealing member 132 can operate as a check valve or one-way valve to prevent backflow into the second tube 114. In other embodiments, the sealing member 132 can be disposed at the junction 116 to prevent backflow of fluid into the second tube 114. The sealing member 132 can be configured to form an air-tight seal at the cleaning system 126 on the second tube 114. The sealing member 132 can allow the visualization device 122 to pass through the cleaning system 126 while maintaining the air-tight seal. If the visualization device 122 is retracted from the cleaning system 126 towards the proximal end 104 of the endotracheal tube 100, the sealing member 132 can reform the air-tight seal once the visualization device 122 is beyond the cleaning system 126. The sealing member 132 can thereby militate against pressurized oxygen from the ventilator flowing through the first tube 112 and past the junction 116 from backflowing into the second tube 114, in operation. It should be appreciated that the sealing means 126 can provide an air-tight seal of the second tube 114 through insertion and use of the endotracheal tube 100.

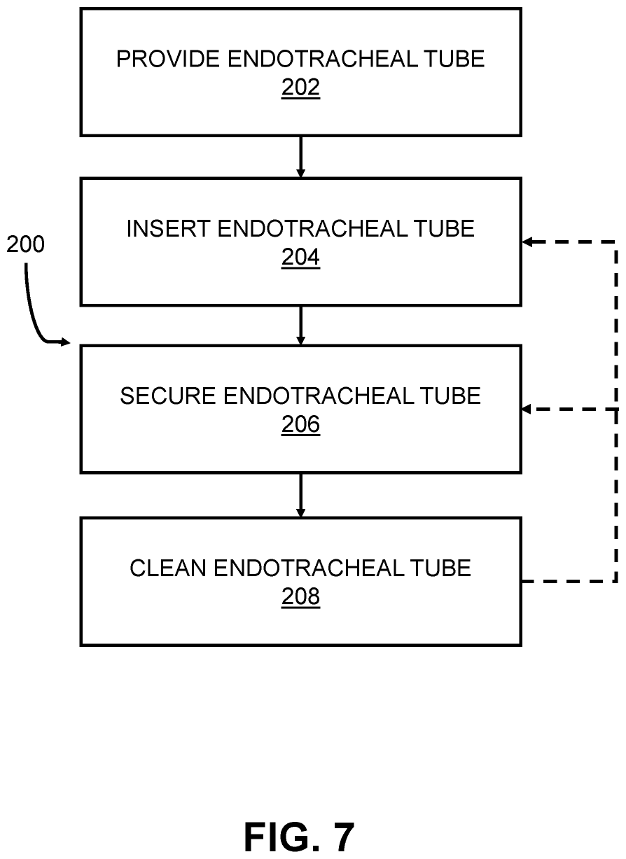

In a further embodiment, for example as shown in FIG. 7, the present disclosure relates to a method 200 for utilizing the endotracheal tube 100. The method 200 can include a step 202 of providing the endotracheal tube 100, as described hereinabove. The endotracheal tube 100 can include the stylet 120 disposed in the first tube 112 and the visualization device 122 disposed through the second tube 114 past the junction 116.

A step 204 of the method 200 can include inserting the distal end 104 of the endotracheal tube 100 into the airway 103 of the patient 101. The flexible tip member 106 and the stylet 120 can be used to navigate the airway 103.

The method 200 can include a step 206 of securing the endotracheal tube 100. The visualization device 122 can be utilized to confirm a proper placement of endotracheal tube 100 at a desired location in the airway. When the endotracheal tube 100 is in the desired location, the inflatable cuff 110 can be inflated to secure the endotracheal tube 100 in place and the stylet 120 can be removed.

A step 208 of the method 200 can include cleaning the visualization device 122, as needed, with the cleaning system 126. During insertion and placement of the endotracheal tube 100, the visualization device 122 can become encumbered with debris. As needed, the visualization device 122 can be retracted toward the proximal end 104 of the endotracheal tube 100. In particular, the visualization device can be retracted back through the junction 116 and through the cleaning system 126. The visualization device 122 can be repeatedly passed into the cleaning system 126, for example, as shown in FIGS. 5-6. The physical agitator 128 and the fluid injection assembly 130 can be used to clear debris from the visualization device 122. It should be appreciated that the cleaning step 208 can be performed, as needed, throughout the method 200. Once cleaned, the visualization device 122 can be advanced toward the distal end 102 of the endotracheal tube 100, as needed.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An endotracheal tube, configured to be inserted in an airway of a patient and cooperate with a ventilator and a visualization device, the endotracheal tube comprising:
a tube body having a distal end configured to be disposed in the airway and a proximal end configured to remain outside the airway, the tube body including a first tube and a second tube, the first tube configured to be operably coupled to the ventilator and the second tube configured to operably receive and have the visualization device advanced therethrough;
a cleaning system disposed in the second tube; and
a flexible tip member disposed on the distal end of the tube body.

2. The endotracheal tube of claim 1, wherein the flexible tip member has a plurality of holes formed therethrough.

3. The endotracheal tube of claim 2, wherein the plurality of holes are arranged in an array across an entire surface of the flexible tip member.

4. The endotracheal tube of claim 1, wherein the tube body includes a junction where the second tube merges into the first tube.

5. The endotracheal tube of claim 4, wherein the second tube extends into the first tube distal to the junction.

6. The endotracheal tube of claim 4, wherein an inflatable cuff is disposed between the distal end of the tube body and the junction.

7. The endotracheal tube of claim 4, wherein a diameter of the tube body distal to the junction is less than a diameter of the tube body proximal to the junction.

8. The endotracheal tube of claim 7, wherein the first tube circumscribes the second tube distal to the junction.

9. The endotracheal tube of claim 4, wherein the cleaning system is disposed in the second tube proximal to the junction.

10. The endotracheal tube of claim 9, wherein the cleaning system includes a physical agitator.

11. The endotracheal tube of claim 10, wherein the physical agitator includes bristles.

12. The endotracheal tube of claim 9, wherein the cleaning system includes a a fluid injection assembly.

13. The endotracheal tube of claim 1, wherein a sealing member is disposed adjacent to the cleaning system on the second tube and provides an air-tight seal of the second tube.

14. The endotracheal tube of claim 1, wherein a stylet is disposed in the first tube through the proximal end.

15. The endotracheal tube of claim 1, further comprising the visualization device, wherein the visualization device is disposed in the second tube at the proximal end.

16. The endotracheal tube of claim 15, wherein the visualization device is a bronchoscope.

17. The endotracheal tube of claim 16, wherein a stylet is disposed in the first tube at the proximal end.

18. The endotracheal tube of claim 1, wherein the flexible tip member has a plurality of holes formed therethrough and the plurality of holes are arranged in an array across an entire surface of the flexible tip member, and the cleaning system includes a physical agitator and a fluid injection port.

19. The endotracheal tube of claim 18, wherein a sealing member is disposed adjacent to the cleaning system on the second tube and provides an air-tight seal of the second tube.

20. A method of utilizing an endotracheal tube, the endotracheal tube configured to be inserted in an airway of a patient and cooperate with a ventilator, the method comprising:
providing the endotracheal tube including a tube body having a distal end configured to be disposed in the airway and a proximal end configured to remain outside the airway, the tube body including a first tube and a second tube, the first tube configured to be operably coupled to the ventilator and a visualization device disposed in the second tube configured to be advanced therethrough; a cleaning system disposed in the second tube; and a flexible tip member disposed on the distal end of the tube body;
inserting the endotracheal tube into the airway;
securing the endotracheal tube at a desired location in the airway; and
cleaning the visualization device with the cleaning system.
